(19) United States
(12) Patent Application Publication
(10) Pub. No.: US 2025/0308515 A1
(43) Pub. Date: Oct. 2, 2025

(54) METHOD, DEVICE, AND COMPUTER-READABLE STORAGE MEDIUM FOR INTELLIGENT INTERACTION

(71) Applicant: Fulian Precision Electronics (Tianjin) Co., Ltd., Tianjin (CN)

(72) Inventor: CHENG-YU WANG, New Taipei (TW)

(21) Appl. No.: 18/962,228

(22) Filed: Nov. 27, 2024

(30) Foreign Application Priority Data
Apr. 1, 2024 (CN) 202410386725.2

(51) Int. Cl. G10L 15/18; A61B 5/024; A61B 5/16; G10L 15/22

(52) U.S. Cl. CPC G10L 15/1815; A61B 5/024; A61B 5/165; G10L 15/22

(57) ABSTRACT

A method for intelligent interaction is provided. The method comprises: obtaining a dialogue content with an interactive object and a first emotional data of the interactive object in a first period; determining a first emotional type according to the first emotional data; activating a first emotional model corresponding to the first emotional type; generating a first response content according to the dialogue content through invoking the first emotional model; and responding to the dialogue content according to the first response content. The first response content is generated based on the dialogue content through the first emotional model, such that the first response content matches the first emotional type.

### CROSS-REFERENCE TO RELATED APPLICATIONS

This application claims priority to Chinese Patent Application No. 202410386725.2 filed on Apr. 1, 2024, in China National Intellectual Property Administration, the contents of which are incorporated by reference herein.

### FIELD

The subject matter herein generally relates to Artificial Intelligence (AI) technology field, and more particularly to a method, device, and computer-readable storage medium for intelligent interaction.

### BACKGROUND

With the development of AI technology, a dialogue content spoken by users may be analyzed through semantic analysis technology by an AI, such that real intentions of the dialogue content may be obtained by the AI, and a response content may be provided according to the real intentions of the dialogue content by the AI. However, the dialogue content is often emotional, such that the response content provided by the AI is difficult to adapt to the emotions of the users.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a flowchart of a method for intelligent interaction provided by an embodiment of the present disclosure.

FIG. 2 is a structural diagram of a device for intelligent interaction provided by an embodiment of the present disclosure.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts have been exaggerated to better show details and features of the present disclosure.

Several definitions that apply throughout this disclosure will now be presented.

The term “coupled” is defined as connected, whether directly or indirectly through intervening components, and is not necessarily limited to physical connections. The connection may be such that the objects are permanently connected or releasably connected. The term “comprising,” when utilized, means “including, but not necessarily limited to”; it specifically indicates open-ended inclusion or membership in the so-described combination, group, series, and the like.

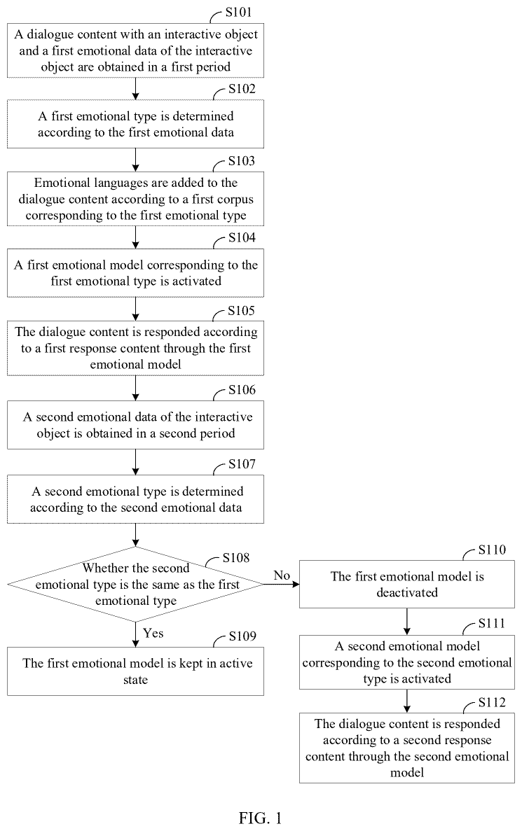

FIG. 1 is a flowchart of a method for intelligent interaction. As shown in FIG. 1, the method includes the following blocks.

At block S101, a dialogue content with an interactive object and a first emotional data of the interactive object are obtained in a first period.

In block S101, the first emotional data may include a physiological data and a behavioral data. The physiological data may include heart rates, skin conductivities, respiratory rates, and facial images. The behavioral data may include limb movement images.

In this embodiment, the dialogue content may be recorded through a microphone in the first period, and the intentions of the dialogue content may be identified through a semantic recognition algorithm, such that the dialogue content may be obtained accurately. The first emotional data may be collected through at least one sensor in the first period. The first period may be set as desired, for example, the first period is 3 minutes.

At block S102, a first emotional type is determined according to the first emotional data.

In this embodiment, a feature parameter may be extracted from the first emotional data through time-domain analysis or frequency-domain analysis or nonlinear fitting, and the first emotional type matched to the feature parameter may be determined according to a preset correspondence between feature parameters and emotional types. The emotional types may be set as desired, for example, the emotional types may include excited emotion, steady emotion, downcast emotion, and tense emotion.

In block S102, the first emotional data may include heart rates, and the heart rates include Low Frequencies (LFs) and High Frequencies (HFs). The LFs are less than a preset frequency threshold, and the HFs are greater than or equal to the preset frequency threshold. The preset frequency threshold may be set as desired, for example, the preset frequency threshold is 0.15 Hz.

In this embodiment, the heart rates are related to activities of sympathetic nerves and activities of parasympathetics. The strength of the activities of sympathetic nerves may be measured through a ratio of a number of the LFs to a number of the HFs. When the ratio increases, meaning the activities of sympathetic nerves stronger. The strength of the activities of parasympathetics may be measured through the number of the HFs. When the number of the HFs increases, meaning the activities of parasympathetics stronger.

When the number of the LFs increases and the number of the HFs decreases, such that the ratio increases, meaning the activities of sympathetic nerves stronger and the activities of parasympathetics weaker, such that the first emotional type is excited emotion.

When the number of the LFs decreases and the number of the HFs increases, such that the ratio decreases, meaning the activities of sympathetic nerves weaker and the activities of parasympathetics stronger, such that the first emotional type is steady emotion.

When the number of the LFs decreases and the number of the HFs decreases, such that the ratio may increase or decrease, meaning the activities of sympathetic nerves unstable and the activities of parasympathetics weaker, such that the first emotional type is downcast emotion.

When the number of the LFs increases and the number of the HFs increases, such that the ratio may increase or decrease, meaning the activities of sympathetic nerves unstable and the activities of parasympathetics stronger, such that the first emotional type is tense emotion.

In another embodiment, the LFs range from 0.04 Hz to 0.15 Hz, and the HFs range from 0.15 Hz to 0.4 Hz.

At block S103, emotional languages are added to the dialogue content according to a first corpus corresponding to the first emotional type.

In this embodiment, the first corpus may be stored locally or in a cloud. The first corpus is used to store the emotional languages related to the first emotional type. The emotional languages may include auxiliary words and adjectives used to express the first emotional type.

In this embodiment, the differences between various emotional types increase through adding emotional languages to the dialogue content, such that the matching degree of emotional models are increased.

At block S104, a first emotional model corresponding to the first emotional type is activated.

In block S104, the first emotional model is used to generate a first response content according to the dialogue content, and the first response content matches the first emotional type.

In this embodiment, the first emotional model may be a random forest model, or a neural network model, or a deep learning model. The first emotional model may be trained based on an emotional training set corresponding to the first emotional type. The emotional training set may include a text content corresponding to the first emotional type.

In this embodiment, when the first emotional model is not activated, it is in dormant state to save system resources and decrease system power consumption. When the first emotional model is activated, it is in active state to response to the dialogue content.

In this embodiment, an activation command is triggered after determining the first emotional type. The activation command is used to activate the first emotional model.

In this embodiment, the first emotional model is automatically loaded, such that omitting a model loading process during the interaction process to improve response efficiency.

At block S105, the dialogue content is responded according to a first response content through the first emotional model.

In this embodiment, the first response content is generated based on the dialogue content through the first emotional model, such that the first response content matches the first emotional type.

At block S106, a second emotional data of the interactive object is obtained in a second period.

In block S106, the second period is after the first period. The second period may be set as desired, for example, the second period is 2 minutes.

In this embodiment, the second emotional data may be collected through at least one sensor to determine whether the first response content is suitable for the emotions of the interactive object in the second period, after responding to the dialogue content.

At block S107, a second emotional type is determined according to the second emotional data.

In block S107, determining the second emotional type may be referred to the relevant description of block S102 and will not be repeated herein.

At block S108, whether the second emotional type is the same as the first emotional type is determined.

When the second emotional type is the same as the first emotional type, meaning the first response content is suitable for the emotions of the interactive object in the second period, and block S109 is implemented.

When the second emotional type is different from the first emotional type, meaning the first response content is not suitable for the emotions of the interactive object in the second period, and block S110-S112 are implemented.

At block S109, the first emotional model is kept in active state.

In this embodiment, when the second emotional type is the same as the first emotional type, the first emotional model is not changed in the second period.

In this embodiment, an emotional model may be in active state or dormant state. One of the multiple emotional models is in active state, while the other emotional models are in dormant state in a certain period.

At Block S110, the first emotional model is deactivated.

In this embodiment, a deactivation command is triggered after determining the second emotional type is different from the first emotional type. The deactivation command is used to deactivate the first emotional model corresponding to the first emotional type. When the first emotional model is deactivated, it is switched to dormant state to save system resources and decrease system power consumption.

At block S111, a second emotional model corresponding to the second emotional type is activated.

In block S111, the second emotional model is used to generate a second response content according to the dialogue content, and the second response content matches the second emotional type.

At block S112, the dialogue content is responded according to a second response content through the second emotional model.

In this embodiment, the second response content is generated based on the dialogue content through the second emotional model, and the second response content matches the second emotional type.

The above description of the present disclosure introduces a method for intelligent interaction. The method may be applied to a device for intelligent interaction.

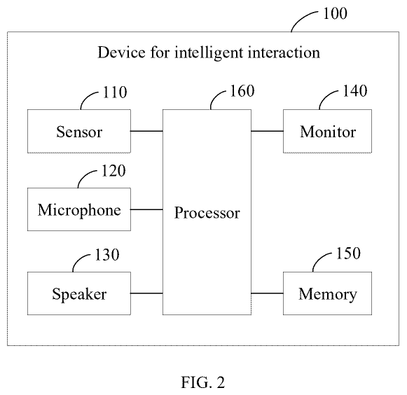

FIG. 2 is a structural diagram of a device for intelligent interaction. As shown in FIG. 2, the device 100 includes a sensor 110, a microphone 120, a speaker 130, a monitor 140, a memory 150, and a processor 160. The processor 160 is electrically connected to the sensor 110, the microphone 120, the speaker 130, the display 140, and the memory 150.

The sensor 110 is used to collect an emotional data of an interactive object.

In this embodiment, the sensor 110 may be a biosensor used to collect a physiological data of an interactive object. The physiological data may include heart rates, skin conductivities, and respiratory rates.

In another embodiment, the sensor 110 may be a camera used to capture facial images and limb motion images of an interactive object.

The microphone 120 is used to record a dialogue content of an interactive object.

The speaker 130 is used to play a response content.

The monitor 140 is used to display a response content.

In this embodiment, the monitor 140 may display a dialogue content of an interactive object recorded through the microphone 120.

The memory 150 is used to store a computer program. The computer program may be executed by the processor 160 to achieve the method described above, which is not repeated here.

The computer program includes computer program codes, and the computer program codes may be in a form of source codes, or object codes, or an executable file.

In this embodiment, the memory 150 may store several emotional models. Every emotional model is used to generate a response content according to a dialogue content, and the response content matches an emotional type.

In this embodiment, the several emotional models may include an excited emotional model, a steady emotional model, a downcast emotional model, and a tense emotional model. The excited emotional model is used to generate a response content according to a dialogue content, the response content matches excited emotion. The steady emotional model is used to generate a response content according to a dialogue content, the response content matches a steady emotion. The downcast emotional model is used to generate a response content according to a dialogue content, the response content matches a downcast emotion. The tense emotional model is used to generate a response content according to a dialogue content, the response content matches a tense emotion.

In another embodiment, the memory 150 may store several corpora, and every corpus is corresponding to an emotional type. A corpus corresponding to excited emotion is used to store emotional languages related to excited emotion. A corpus corresponding to steady emotion is used to store emotional languages related to steady emotion. A corpus corresponding to downcast emotion is used to store emotional languages related to downcast emotion. A corpus corresponding to tense emotion is used to store emotional languages related to tense emotion.

In some embodiments, the memory 150 may be an internal storage unit of the device 100, such as a hard disk or a memory of the device 100. In other embodiments, the memory 150 may also be an external storage device of the device 100, such as a plug-in hard disk, a smart memory card (SMC), a secure digital (SD) card, a flash card. Further, the memory 150 may also include both an internal storage unit of the device 100 and an external storage device. The memory 150 is used to store an operating system, a disclosure program, and other programs, such as the program code of the computer program. The memory 150 may also be used to temporarily store data that has been output or is to be output.

The processor 160 may be, but is not limited to, a central processing unit (CPU), a general-purpose processor, a digital signal processor (DSP), an application-specific integrated circuit (ASIC), a field-programmable gate array (FPGA) or another programmable logic device, a discrete gate or transistor logic device, a discrete hardware component. The general-purpose processor may be a microprocessor or any conventional processor.

In some embodiments, the device 100 may include an input module. The input module is used to receive a dialogue content in a form of text in a certain period. The input module may include a touch screen.

The above description of the present disclosure introduces a device for intelligent interaction. The device may include at least one sensor, at least one microphone, at least one speaker, a memory, and at least one processor.

In one embodiment, the device may be applied in a system for intelligent customer service. For example, the device is integrated into a robot for intelligent customer service, such that the robot is used to obtain an emotional data of a customer, identify an emotional type, and invoke an emotional model corresponding to the emotional type to generate a response content. The response content is suitable for the emotions of the customer.

In another embodiment, the device may be applied in a social media tool for emotion analysis. For example, the device is integrated into a computer, the computer is used to obtain an emotional data on a social media, identify an emotional type, and invoke an emotional model corresponding to the emotional type to generate a response content. The response content is suitable for the emotions of the public on the social media.

The present disclosure further provides a computer-readable storage medium, the computer-readable storage medium is used to store a computer program. The computer program may be executed by a processor to achieve the method described above, which is not repeated here.

The computer-readable medium may include a read-only memory (ROM), a random access memory (RAM), a USB flash drive, a mobile hard disk, a magnetic disk or an optical disk.

The above description only describes embodiments of the present disclosure, and is not intended to limit the present disclosure, various modifications and changes can be made to the present disclosure. Any modifications, equivalent substitutions, improvements, etc. made within the spirit and scope of the present disclosure are intended to be included within the scope of the present disclosure.

What is claimed is:

1. A method for intelligent interaction, comprising:
obtaining a dialogue content with an interactive object and a first emotional data of the interactive object in a first period;
determining a first emotional type according to the first emotional data;
activating a first emotional model corresponding to the first emotional type;
generating a first response content according to the dialogue content through invoking the first emotional model; and
responding to the dialogue content according to the first response content.

2. The method of claim 1, further comprising:
obtaining a second emotional data of the interactive object in a second period;
determining a second emotional type according to the second emotional data;
when the second emotional type is different from the first emotional type, activating a second emotional model corresponding to the second emotional type, and generating a second response content according to the dialogue content through invoking the second emotional model; and
responding to the dialogue content according to the second response content.

3. The method of claim 2, further comprising:
when the second emotional type is different from the first emotional type, deactivating the first emotional model.

4. The method of claim 2, further comprising:
when the second emotional type is the same as the first emotional type, keeping the first emotional model in active state.

5. The method of claim 1, before activating a first emotional model corresponding to the first emotional type, the method further comprising:
adding emotional languages to the dialogue content according to a first corpus corresponding to the first emotional type.

6. The method of claim 1, determining a first emotional type according to the first emotional data comprising:
when the first emotional data includes heart rates, determining a first emotional type according to the heart rates;
wherein the heart rates include low frequencies and high frequencies, the low frequencies are less than a preset frequency threshold, and the high frequencies are greater than or equal to the preset frequency threshold.

7. The method of claim 6, determining a first emotional type according to the heart rates comprising:
when the number of the low frequencies increases and the number of the high frequencies decreases, determining the first emotional type is excited emotion;
when the number of the low frequencies decreases and the number of the high frequencies increases, determining the first emotional type is steady emotion;
when the number of the low frequencies decreases and the number of the high frequencies decreases, determining the first emotional type is downcast emotion;
when the number of the low frequencies increases and the number of the high frequencies increases, determining the first emotional type is tense emotion.

8. The method of claim 6, wherein:
the low frequencies range from 0.04 Hz to 0.15 Hz, and the high frequencies range from 0.15 Hz to 0.4 Hz.

9. The method of claim 1, before activating a first emotional model corresponding to the first emotional type, the method further comprising:
training the first emotional model based on an emotional training set;
wherein the emotional training set is corresponding to the first emotional type.

10. A device for intelligent interaction, the device comprising: a memory;
at least one sensor;
at least one microphone;
at least one speaker;
at least one processor; and
at least one computer program stored in the memory, which when executed by the at least one processor, cause the at least one processor to:
obtain a dialogue content with an interactive object in a first period through invoking the at least one microphone;
obtain a first emotional data of the interactive object in the first period through invoking the at least one sensor;
determine a first emotional type according to the first emotional data;
activate a first emotional model corresponding to the first emotional type;
generate a first response content according to the dialogue content through invoking the first emotional model; and
respond to the dialogue content according to the first response content through invoking the at least one speaker.

11. The device of claim 10, the at least one processor is further configured to:
obtain a second emotional data of the interactive object in a second period through invoking the at least one sensor;
determine a second emotional type according to the second emotional data;
when the second emotional type is different from the first emotional type, activate a second emotional model corresponding to the second emotional type, and generate a second response content according to the dialogue content through invoking the second emotional model; and
respond to the dialogue content according to the second response content through invoking the at least one speaker.

12. The device of claim 11, the at least one processor is further configured to:
when the second emotional type is different from the first emotional type, deactivate the first emotional model.

13. The device of claim 11, the at least one processor is further configured to:
when the second emotional type is the same as the first emotional type, keep the first emotional model in active state.

14. The device of claim 10, before activating a first emotional model corresponding to the first emotional type, the at least one processor is further configured to:
add emotional languages to the dialogue content according to a first corpus corresponding to the first emotional type.

15. The device of claim 10, determining a first emotional type according to the first emotional data comprising:
when the first emotional data includes heart rates, determining a first emotional type according to the heart rates;
wherein the heart rates include low frequencies and high frequencies, the low frequencies are less than a preset frequency threshold, and the high frequencies are greater than or equal to the preset frequency threshold.

16. The device of claim 15, determining a first emotional type according to the heart rates comprising:
when the number of the low frequencies increases and the number of the high frequencies decreases, determining the first emotional type is excited emotion;
when the number of the low frequencies decreases and the number of the high frequencies increases, determining the first emotional type is steady emotion;
when the number of the low frequencies decreases and the number of the high frequencies decreases, determining the first emotional type is downcast emotion;
when the number of the low frequencies increases and the number of the high frequencies increases, determining the first emotional type is tense emotion.

17. The device of claim 15, wherein:
the low frequencies range from 0.04 Hz to 0.15 Hz, and the high frequencies range from 0.15 Hz to 0.4 Hz.

18. The device of claim 10, before activating a first emotional model corresponding to the first emotional type, the at least one processor is further configured to:
train the first emotional model based on an emotional training set;
wherein the emotional training set is corresponding to the first emotional type.

19. The device of claim 10, the device further comprising: a monitor;
the monitor is used to display a response content.

20. A computer-readable storage medium for intelligent interaction, the computer-readable storage medium stores a computer program, which when executed by at least one processor, cause the at least one processor to:
obtain a dialogue content with an interactive object in a first period through invoking at least one microphone;
obtain a first emotional data of the interactive object in the first period through invoking at least one sensor;
determine a first emotional type according to the first emotional data;
activate a first emotional model corresponding to the first emotional type;
generate a first response content according to the dialogue content through invoking the first emotional model; and
respond to the dialogue content according to the first response content through invoking at least one speaker.
